(19) United States
(12) Patent Application Publication
(10) Pub. No.: US 2003/0167186 A1
(43) Pub. Date: Sep. 4, 2003

(54) Computer-aided method for documenting a medical finding

(76) Inventors: Tilo Christ, Erlangen (DE); Heinz Prihoda, Heroldsberg (DE); Volker Schmidt, Erlangen (DE); Siegfried Schneider, Erlangen (DE); Hans-Dieter Schuell, Weisendorf (DE); Werner Striebel, Schwarzenbruck (DE); Gudrun Zahlmann, Neumarkt (DE)

Correspondence Address: HARNESS, DICKEY & PIERCE, P.L.C., P.O.BOX 8910, RESTON, VA, 20195, US

(21) Appl. No.: 10/317,124

(22) Filed: Dec. 12, 2002

(30) Foreign Application Priority Data
Dec. 12, 2001 (DE) 10161112.9

(51) Int. Cl. A61B 5/00; G06F 17/60

(52) U.S. Cl. 705/2; 600/300

(57) ABSTRACT

A computer-aided method is for documenting a medically insignificant finding. By way of example, a display device on the computer system is used to display an electronic form suitable for the documentation to a doctor. The doctor then need activate only a single input element on the computer system, so that the activated input element is used to complete the electronic form automatically using prescribed data which are stored on the computer system and associated with an insignificant medical finding. A computer-aided method is also for documenting a medical finding. For this method, a display device on a computer system is used to display an electronic form suitable for the documentation to the doctor, for example. When the electronic form has been completed using data associated with the medical finding, a single input element on the computer system is activated, and the activated input element automatically prompts performance of at least one action by the computer system.

The present application hereby claims priority under 35 U.S.C. §119 on German patent application number 10161112.9 filed Dec. 12, 2001, the entire contents of which are hereby incorporated herein by reference.

### FIELD OF THE INVENTION

The invention generally relates to methods for documenting a medical finding using a computer system.

### BACKGROUND OF THE INVENTION

Following examination of a patient, a doctor examining the patient generally documents the result of the examination using a suitable form, for example. The result of the examination is also referred to as the medical finding for the patient. Increasingly, the findings are also being documented electronically by virtue of the doctor completing forms stored on a computer, for example, using the computer. The medical finding is also documented if the examination shows that the patient is healthy. Such a finding is also referred to as a normal finding or insignificant (nonsignificant) finding.

Particularly in the case of serial examinations, such as serial X-ray examinations, serial pulmonary examinations, mammography screenings or eye fundus screenings, pediatric checkups or cancer checkups, a relatively large number of insignificant findings are obtained. Thus, the doctor has to document a large number of findings which essentially have the same content. Since the insignificant findings essentially have the same content, the doctor has to complete a large number of forms with essentially the same information for the serial examination.

When the doctor has completed the form, he generally has to print the form comprising the medical finding and needs to sign it, or he needs to sign the completed form stored on the computer digitally, send the completed form to other people, etc. This can be relatively time-consuming, in particular, since he needs to initiate a plurality of processes in succession. Furthermore, the actions just described also need to be performed by the doctor when documenting a pathological finding, that is to say when a finding for a sick patient is involved.

### SUMMARY OF THE INVENTION

An object of an embodiment of the invention is therefore to provide prerequisites for simplified recording of an insignificant finding. Another object of an embodiment of the invention is to provide prerequisites for simpler processing of an insignificant finding or a pathological finding.

The first object may be achieved by a computer-aided method for documenting an insignificant finding, having the following method steps:

a) an electronic form suitable for the documentation is displayed using a display device on a computer system,

b) a single input element on the computer system is activated, and

c) activation of the input element prompts automatic completion of the electronic form using prescribed data which are stored on the computer system and are associated with an insignificant finding.

As already described above, when documenting a medical finding, the doctor generally completes a preproduced form which is associated with the type of examination and, by way of example, is in the form of a checklist, so that the doctor answers all the questions required for the medical finding. In the case of insignificant findings for various patients for whom at least similar examinations have been carried out, these forms and the answers to the questions on the forms are essentially identical. Thus, the doctor needs to complete a large number of forms with essentially the same information.

If the doctor now wishes to complete such a form, and the form is associated with a patient having an insignificant finding, an embodiment of the invention requires him merely to activate the input element, and the questions on the form are answered using data stored on the computer system. Consequently, when recording the insignificant finding, the doctor merely needs to activate an input element in order to obtain the completed form corresponding to the insignificant finding. There is thus no need for time-consuming, essentially identical completion of a number of forms.

In line with one embodiment of the invention, a window and/or a field is/are opened in the form and is/are used to request activation of the input element. If the doctor views the form using the display device on the computer system, the window or the field can be opened in the form being shown or outside of the form being shown.

In accordance with another variant of the invention, the input element is a computer mouse or a keyboard on a computer system, and the activation of the input element is the pressing of a key on the computer mouse or on the keyboard. It is therefore possible, in line with one particularly preferred variant of the invention, for the field to be clicked on using the computer system's computer mouse, and for this automatically to complete the form using the prescribed data stored on the computer system. The window for requesting activation of the input element can also comprise a field which initiates automatic completion of the form, particularly as a result of clicking.

In line with another embodiment of the invention, activation of the input element prompts automatic performance of at least one further prescribed action by the computer system. In line with one particularly preferred variant of the invention, the further action is digital signing of the completed electronic form, encryption of the completed electronic form, transmission of the completed electronic form to at least one receiver, archiving of the completed electronic form, invoicing or debiting from an account. The actions just cited are often performed after a medical finding has been recorded, that is to say even when an insignificant finding has been recorded. The inventive method means that these actions do not need to be initiated separately, however, but rather are performed automatically on account of the activation of the input element.

The second object of the invention may be achieved by a computer-aided method for documenting a medical finding, having the following method steps:

a) an electronic form suitable for the documentation is displayed using a display device on a computer system,

b) the electronic form is completed using data associated with the medical finding,

c) a single input element on the computer system is activated, and

d) activation of the input element prompts automatic performance of at least one action by the computer system.

As already described above, when a medical finding has been recorded, a doctor often needs to perform a number of further actions, such as digital signing of the completed electronic form, encryption of the completed electronic form, transmission of the completed electronic form to at least one receiver, archiving of the completed electronic form, invoicing or debiting from an account. By activating a single input element, an embodiment of the invention involves these actions being performed automatically, which saves time for the doctor or the person who completes the electronic form associated with the medical finding.

Depending on whether the finding is an insignificant finding or a pathological finding, different actions may need to be performed. Hence, by way of example, it may be necessary only in the event of a pathological finding to notify a health office or specialist for the purpose of further treatment of the patient associated with the pathological finding. This may be the case with a serial pulmonary examination, for example. For this reason, one preferred embodiment of the invention involves checking whether the data associated with the finding are associated with an insignificant finding or a pathological finding, and at least one varying action being performed automatically on the basis of the check.

In accordance with one embodiment of the invention, the input element is a computer mouse or a keyboard on the computer system, and the activation of the input element is the pressing of a key on the computer mouse or on the keyboard. In addition, in line with another variant of the invention, a window and/or a field is/are opened in the electronic form and is/are used to request activation of the input element. Thus, in line with one particularly preferred embodiment of the invention, the field can be clicked on using the computer mouse, and this can perform the action. The action can thus be initiated by a single computer mouse click.

One advantage of the inventive methods is that complex procedures can be handled particularly with a mouse click or by pressing a single key. In addition, it can be advantageous if information is displayed simultaneously in a plurality of windows, so that a user, that is to say the doctor recording the medical finding, for example, sees all the necessary data for the actual confirmation procedure. This is necessary for digital signing, for example, since the document to be signed needs to be displayed as a whole.

The inventive methods can be used, in particular, for every form of electronic finding, for telemedical services, for a “second opinion”, for a screening or for in-house information systems.

The inventive methods result in significant speeding-up of the documentation procedure.

### BRIEF DESCRIPTION OF THE DRAWINGS

Exemplary embodiments are illustrated by way of example in the appended schematic drawings, in which:

FIG. 1 shows a scenario illustrating the inventive methods,

FIG. 2 shows an electronic form,

FIG. 3 shows a computer keyboard,

FIG. 4 shows a completed electronic form documenting an insignificant finding, and

FIG. 5 shows a completed electronic form documenting a pathological finding.

### DETAILED DESCRIPTION OF THE PREFERRED EMBODIMENTS

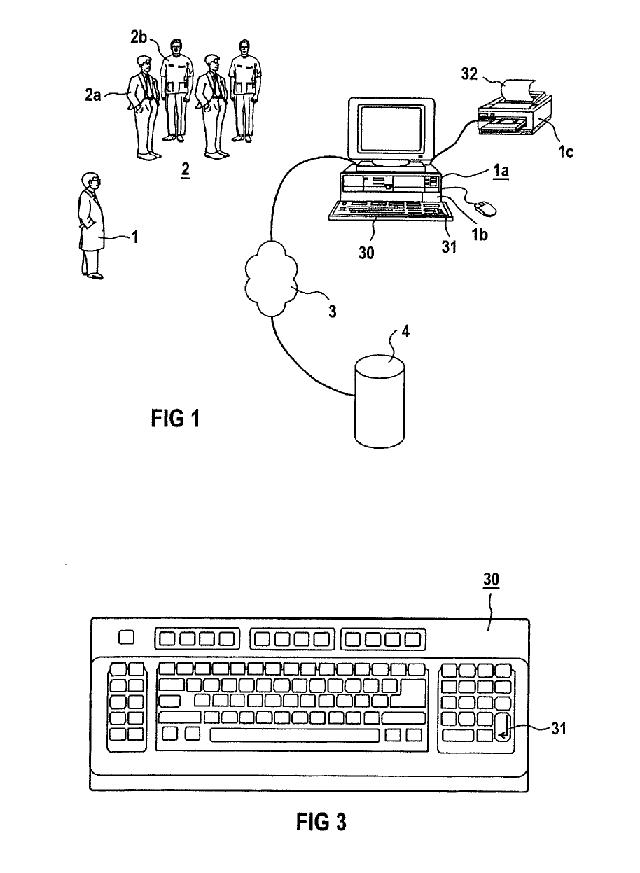

FIG. 1 shows a doctor 1 performing a serial pulmonary examination on a plurality of patients 2. For the serial pulmonary examination, the doctor 1 produces, for each of the patients 2, a respective X-ray (not shown in FIG. 1) using an X-ray machine (which is likewise not shown in FIG. 1). In addition, the doctor 1 performs a blood test on each of the patients 2.

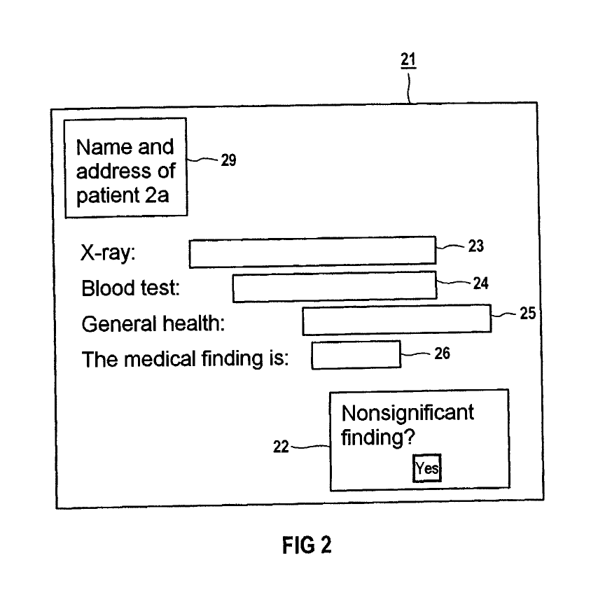

To document the examination result for each of the patients 2, the doctor 1 records a medical finding for each of the patients 2. In the case of the present exemplary embodiment, the doctor 1 prepares the medical findings using a computer 1a. To this end, the computer 1a stores a suitable computer program which is designed to display an electronic form 21, which is shown in FIG. 2, to the doctor 1 on the screen of the computer 1a when the computer program is called. The electronic form 21 includes a field 29 into which the doctor 1 enters the name and address of that patient among the patients 2 for whom he currently wishes to record a medical finding. In addition, the electronic form 21 includes fields 23, 24 and 25 into which the doctor 1 enters any significance regarding the X-ray, regarding the blood test and regarding the general health of the patient for whom the medical finding is being recorded. On the basis of these entries, the doctor 1 can decide whether the medical finding is insignificant or pathological, and he enters this into the field 26.

In case of the present exemplary embodiment, the doctor 1 records a medical finding for a patient 2a among the patients 2. When the doctor 1 has entered the name and address of the patient 2a into the electronic form 21, the computer program automatically opens a field 22 in the form 21, this field 22 asking the doctor 1 whether the present medical finding is an insignificant finding. In the case of the present exemplary embodiment, the X-ray taken from the patient 2a and the blood test performed on the patient 2a lead the doctor 1 to the conclusion that the patient 2a is healthy, and hence that an insignificant finding can be assigned to him. The doctor 1 therefore presses the “enter key” key 31, for example, on the keyboard 30 on the computer 1a. The keyboard 30 is shown in more detail in FIG. 3.

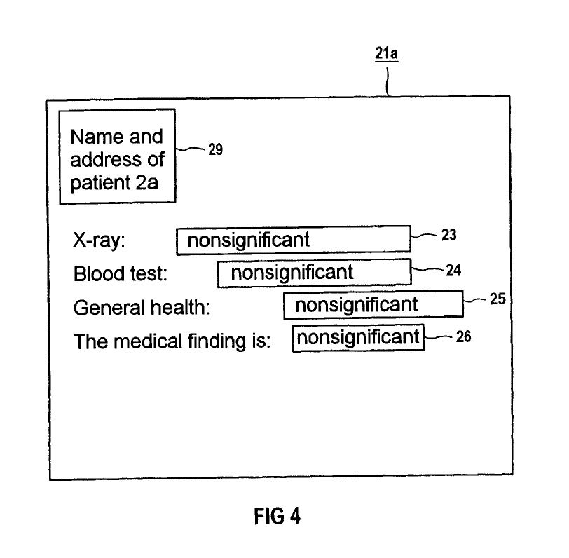

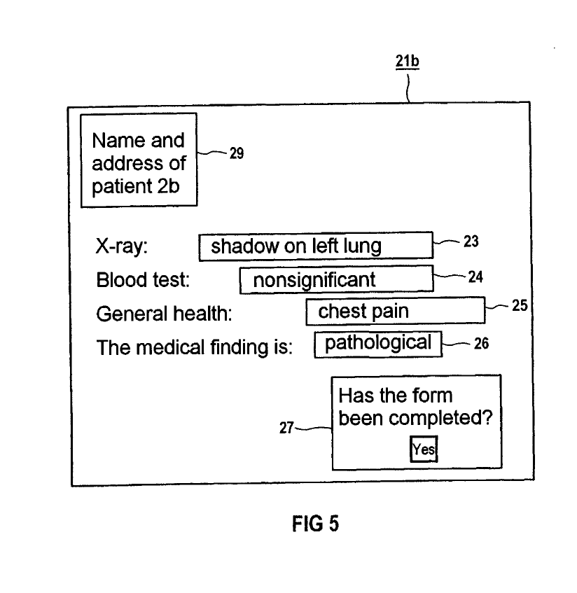

When the “enter” key 31 has been pressed, the computer program automatically completes the electronic form 21 such that an insignificant finding for the patient 2a is documented. The computer program therefore automatically completes the fields 23, 24, 25 and 26 with the word “insignificant”, as shown in FIG. 4. The completed electronic form has been provided with the reference symbol 21a in FIG. 4.

When the “enter” key 31 is pressed on the keyboard 30 on the computer 1a, the computer program does not just automatically complete the electronic form 21; the computer program also preferably automatically provides the completed electronic form 21a with an electronic signature for the doctor 1, encrypts the completed electronic form 21a using generally known methods, stores the encrypted completed electronic form 21a in a memory 1b in the computer 1a and automatically transmits it to a central database 4, which is associated with a health office in the case of the present exemplary embodiment. In another preferred embodiment, the computer program performs at lest one of the aforementioned functions.

So that the completed electronic form 21a can be transmitted to the database 4, the computer 1a and the database 4 are connected to a telecommunication network 3 in the case of the present exemplary embodiment. In addition, the computer program automatically prompts a printer 1c connected to the computer 1a to print an invoice 32 for recording the insignificant finding for the patient 2a. The invoice 32 is intended for the health insurance company of the patient 2a.

Subsequently, the doctor 1 wishes to record a medical finding for a patient 2b among the examined patients 2. He therefore calls up a new uncompleted electronic form 21 and enters the name and address of the patient 2b into the field 29. The computer program then automatically opens the field 22 in the electronic form 21 again.

In the case of the present exemplary embodiment, the X-ray from the patient 2b has a shadow. In addition, the patient 2b is complaining of chest pains. The doctor therefore wishes to issue a pathological finding.

Instead of pressing the “enter” key 31 on the keyboard 30 on the computer 1a, the doctor 1 therefore enters the comment “shadow on left lung” into the field 23. The computer program then automatically closes the field 22. Since the blood test on the patient 2b was insignificant in the case of the present exemplary embodiment, the doctor 1 enters the word “insignificant” into the field 24. In order to document the chest pain in the patient 2b, the doctor 1 enters the comment “chest pain” into the field 25. In addition, the doctor 1 enters the word “pathological” into the field 26 in order to document the pathological finding for the patient 2b in this manner.

When the doctor 1 has completed the fields 23, 24, 25 and 26, the computer program automatically opens a field 27 in the completed electronic form. The completed electronic form is shown in FIG. 5 and has been provided with the reference symbol 21b. The field 27 asks the doctor 1 whether he has completed the electronic form 21. Since the doctor 1 does not wish to make any corrections to the completed electronic form 21b, he presses the “enter” key 31 on the keyboard 30 on the computer 1a.

The computer program then automatically provides the completed electronic form 21b with an electronic signature from the doctor 1, encrypts the completed electronic form 21b, stores the encrypted completed electronic form 21b in the memory 1b in the computer 1a and automatically transmits it to the central database 4 at the health office. In addition, the computer program automatically prompts the printer 1c to print an invoice for recording the pathological finding for the patient 2b. The invoice is intended for the health insurance company of the patient 2b.

It should be noted that the exemplary embodiments described are to be understood merely by way of example. For example, another key instead of the enter key may be designated and used in place of the “enter” key to trigger the program, including but not limited to mouse click or key, a touch panel key, a wireless and/or remote key or any other type of computer input key or device.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A computer-aided method for documenting an insignificant finding, comprising the steps of:
a) displaying an electronic form, suitable for documentation, using a display device of a computer system;
b) activating a single input element on the computer system; and
c) prompting, via activation of the input element, automatic completion of the electronic form using prescribed data stored on the computer system and associated with an insignificant finding.

2. The method as claimed in claim 1, wherein at least one of a field and a window is opened in the electronic form, to request activation of the input element.

3. The method as claimed in claim 1, wherein the input element is at least one of a computer mouse and a keyboard on the computer system, and wherein the activation of the input element includes pressing of a key on at least one of the computer mouse and the keyboard.

4. The method as claimed in claim 3, wherein the field is clicked on using the computer mouse, and this automatically completes the electronic form using the prescribed data stored on the computer system.

5. The method as claimed in claim 1, wherein activation of the input element prompts automatic performance of at least one further prescribed action by the computer system.

6. The method as claimed in claim 5, wherein the further action includes at least one of digital signing of the completed electronic form, encryption of the completed electronic form, transmission of the completed electronic form to at least one receiver, archiving of the completed electronic form, and invoicing or debiting from an account.

7. A computer-aided method for documenting a medical finding, comprising the steps of:
a) displaying an electronic form, suitable for documentation, using a display device on a computer system;
b) completing the electronic form using data associated with the medical finding;
c) activating a single input element on the computer system; and
d) prompting, via activation of the input element, automatic performance of at least one action by the computer system.

8. The method as claimed in claim 7, wherein the action includes at least one of digital signing of the completed electronic form, encryption of the completed electronic form, transmission of the completed electronic form to at least one receiver, archiving of the completed electronic form, and invoicing or debiting from an account.

9. The method as claimed in claim 7, further comprising checking whether the data associated with the finding are associated with at least one of an insignificant finding and a pathological finding, and performing at least one varying action, automatically, on the basis of the check.

10. The method as claimed in claim 7, wherein the input element is at least one of a computer mouse and a keyboard on the computer system, and the activation of the input element includes the pressing of a key on at least one of the computer mouse and on the keyboard.

11. The method as claimed in claim 7, wherein at least one of a field and a window is opened in the electronic form, to request activation of the input element.

12. The method as claimed in claim 11, wherein the field is clicked on using the computer mouse, to perform the action.

13. The method as claimed in claim 2, wherein the input element is at least one of a computer mouse and a keyboard on the computer system, and wherein the activation of the input element includes pressing of a key on at least one of the computer mouse and the keyboard.

14. The method as claimed in claim 13, wherein the field is clicked on using the computer mouse, and this automatically completes the electronic form using the prescribed data stored on the computer system.

15. The method as claimed in claim 2, wherein activation of the input element prompts automatic performance of at least one further prescribed action by the computer system.

16. The method as claimed in claim 15, wherein the further action includes at least one of digital signing of the completed electronic form, encryption of the completed electronic form, transmission of the completed electronic form to at least one receiver, archiving of the completed electronic form, and invoicing or debiting from an account.

17. The method as claimed in claim 3, wherein activation of the input element prompts automatic performance of at least one further prescribed action by the computer system.

18. The method as claimed in claim 17, wherein the further action includes at least one of digital signing of the completed electronic form, encryption of the completed electronic form, transmission of the completed electronic form to at least one receiver, archiving of the completed electronic form, and invoicing or debiting from an account.

19. The method as claimed in claim 4, wherein activation of the input element prompts automatic performance of at least one further prescribed action by the computer system.

20. The method as claimed in claim 19, wherein the further action includes at least one of digital signing of the completed electronic form, encryption of the completed electronic form, transmission of the completed electronic form to at least one receiver, archiving of the completed electronic form, and invoicing or debiting from an account.

21. The method as claimed in claim 8, further comprising checking whether the data associated with the finding are associated with at least one of an insignificant finding and a pathological finding, and performing at least one varying action, automatically, on the basis of the check.

22. The method as claimed in claim 8, wherein the input element is at least one of a computer mouse and a keyboard on the computer system, and the activation of the input element includes the pressing of a key on at least one of the computer mouse and on the keyboard.

23. The method as claimed in claim 8, wherein at least one of a field and a window is opened in the electronic form, to request activation of the input element.

24. The method as claimed in claim 23, wherein the field is clicked on using the computer mouse, to perform the action.

25. An apparatus operable to perform the method of claim 1.

26. An apparatus operable to perform the method of claim 7.

27. A computer program adapted, when read by a computer, to cause said computer to perform the method of claim 1.

28. A computer program adapted, when read by a computer, to cause said computer to perform the method of claim 7.

29. A computer system for documenting an insignificant finding, comprising:
display means for displaying an electronic form, suitable for documentation;
means for activating a single input element on the computer system; and
means for prompting, via activation of the input element, automatic completion of the electronic form using prescribed data stored on the computer system and associated with an insignificant finding.

30. A computer system for documenting a medical finding, comprising:
display means for displaying an electronic form, suitable for documentation;
means for completing the electronic form using data associated with the medical finding;
means for activating a single input element on the computer system; and
means for prompting, via activation of the input element, automatic performance of at least one action by the computer system.
